(19) United States
(12) Patent Application Publication
(10) Pub. No.: US 2007/0093906 A1
(43) Pub. Date: Apr. 26, 2007

(54) Nucleus implant and method

(75) Inventors: Robert Garryl Hudgins, Burnsville, MN (US); Hugh D. Hestad, Edina, MN (US)

Correspondence Address: WOOD, HERRON & EVANS (ZIMMER SPINE), 2700 CAREW TOWER, 441 VINE STREET, CINCINNATI, OH, 45202, US

(73) Assignee: Zimmer Spine, Inc., Minneapolis, MN (US)

(21) Appl. No.: 11/259,518

(22) Filed: Oct. 26, 2005

(51) Int. Cl. A61F 2/44

(52) U.S. Cl. 623/17.16; 623/17.12

(57) ABSTRACT

The present invention provides intervertebral disc nucleus inserts that may fully or partially replace the natural, or native, intervertebral nucleus. The present invention may include a hydrogel bag outer body that includes an interior cavity for introducing a spiral implant device. The hydrogel bag may be introduced through a cannula into the intervertebral space after a cavity of a desired shape and size has been cleared. The combination of the spiral implant resting configuration and the hydrogel bag substantially fills the cavity. The combination of the spiral implant and the hydrogel bag may improve upon 1) implant expulsion or extrusion; 2) implant sizing; 3) implant conformity; and 4) reduction or prevention of bone edema. In further embodiments the hydrogel bag may include an inner and outer layer or two complimentary bags joined into one structure to better distribute the load between the vertebral endplates.

### TECHNICAL FIELD

The present invention is related to spinal stabilization devices. More particularly, the present invention relates to devices and systems for addressing back pain originating in the disc.

### BACKGROUND

The spinal column is a highly complex system of bones and connective tissues that provides support for the body and protects the delicate spinal cord and nerves. The spinal column includes a series of vertebrae stacked one on top of the other, each vertebral body including an inner or central portion of relatively weak cancellous bone and an outer portion of relatively strong cortical bone. Situated between each vertebral body is an intervertebral disc that cushions and dampens compressive forces experienced by to the spinal column. A vertebral canal containing the spinal cord and nerves is located behind the vertebral bodies.

The bones and connective tissue of an adult human spinal column consists of more than 20 discrete bones coupled sequentially to one another by a tri-joint complex which consist of an anterior disc and the two posterior facet joints, the anterior discs of adjacent bones being cushioned by cartilage spacers referred to as intervertebral discs. The intervertebral disc is made up of a strong outer ring called the annulus (i.e., annulus fibrosus) which is attached to the intervertebral bodies through collagen fibers and a central nucleus (i.e., nucleus pulposus). In spite of these complexities the spine is a highly flexible structure capable of a high degree of curvature and twist in nearly every direction.

There are many types of spinal column disorders including scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in a lumbar or cervical spine) and other disorders caused by abnormalities, disease, or trauma, such as ruptured or slipped discs, degenerative disc disease, fractured vertebra, and the like. In addition intervertebral discs are subject to various types of injury, degeneration and disease. Painful disc syndromes can develop due to the destruction of the intervertebral disc structure. Patients that suffer from such conditions usually experience extreme and debilitating pain, as well as diminished nerve function.

These spinal pathologies limit the range of motion or threaten the critical elements of the nervous system housed within the spinal column. A variety of systems have been disclosed in the art that achieve immobilization by implanting artificial assemblies in or on the spinal column. One of the most common surgical interventions today is arthrodesis, or spine fusion, of one or more motion segments. Clinical success varies considerably, depending upon technique and indications, and consideration must be given to the concomitant risks and complications. For example, it has been shown that spine fusion decreases function by limiting the range of motion for patients in flexion, extension, rotation, and lateral bending. Furthermore, it has been shown that spine fusion creates increased stresses and accelerated degeneration of adjacent non-fused motion segments. Also, the fusion device, whether artificial or biological, may migrate out of the fusion site.

Another surgical intervention includes removing some or all of the intervertebral disc and is called nuclectomy. Nuclectomy may also be referred to as discectomy. When a nucleus implant is placed during a nuclectomy, it may further be referred to as nucleoplasty. One implant that may be inserted during nucleoplasty is a spiral implant. A spiral implant is an elongated elastic body that forms a spiral in the force free state. See, for example, U.S. Pat. Nos. 5,919,235, 6,165,218 and 6,660,037, which are incorporated herein by reference for all that they teach and disclose. The spiral implant can be placed in the inner space of the intervertebral disc through a small opening and utilized as an intervertebral prosthesis. One particular problem with such an implant device, however, is that the spiral may be prone to expulsion or extrusion from the nucleus after implantation. In addition, the spiral implant may not evenly distribute the force over the entire intervertebral space. Other types of implants may also be made from elastic or deformable bodies. Such implants may include various plastics or gel type materials that are implanted in the intervertebral space. Such implants may also have problems with being extruded after implantation. Moreover, such implants may not have the mechanical strength of solid implants.

### SUMMARY

The present invention includes an outer body bag around a spiral nucleus device, the combination utilized in the disc space as an intervertebral prosthesis or implant.

The present invention provides nucleus implants with improved cavity fits and improved resistance to expulsion. The improved cavity fit may also contribute to mechanically reducing inflammatory response.

One embodiment of the present invention includes a spinal stabilization with a bag, the bag for insertion into a cavity formed by the removal of a desired amount of a nucleus of an intervertebral disc and a spiral implant for insertion into the bag after the bag is inserted into the cavity.

Another embodiment of the present invention is a method for replacing a damaged intervertebral disc including the steps of removing a desired portion of a nucleus of the intervertebral disc to form a cavity, inserting a PVA hydrogel bag into the cavity, the PVA hydrogel bag including a wall forming an interior and an opening, inserting a spiral implant into the interior of the PVA hydrogel bag through the opening, and closing the opening of the PVA hydrogel.

Another embodiment includes a method for replacing nucleus of an intervertebral disc, said method including removing a degenerated or damaged intervertebral disc nucleus from a patient thereby creating a cavity within the annulus fibrosus, inserting a compressible prosthetic bag into the cavity using a cannula, the bag including an inner cavity and an opening, placing one or more bodies into the bag, adding a spiral implant into the bag, and closing the opening using a closing mechanism.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a top plan view of an apparatus of the present invention inserted into the spine.

FIG. 2 is a top plan view of a spiral implant for utilization in the present invention.

FIG. 3 is a side plan view of FIG. 1.

FIG. 4. is a side view of a cavity formed in the intervertebral disk

FIG. 5 is a top plan view of a balloon catheter being inserted into the cavity of FIG. 4.

FIG. 6 is a side plan view of a hydrogel bag being inserted into the cavity of the Hydrogel bag of FIG. 4.

FIG. 7 is a side plan view of a spiral implant being inserted into the hydrogel bag of FIG. 4.

FIG. 8 is a perspective view of a hydrogel bag with closure ties.

FIG. 9 is a cross sectional perspective view of an alternative embodiment of the present invention.

FIG. 10 is a top plan view of the alternative embodiment of FIG. 9.

FIG. 11 is a top plan view of the alternative embodiment of FIG. 9 with the spiral implant partially inserted.

FIG. 12 is a top plan view of another alternative embodiment of the present invention.

FIG. 13a is a top plan view of an alternative embodiment with a spar across the inner body.

FIG. 13b is a side perspective view of an alternative embodiment with spars situated on an external portion of the inner body.

FIG. 13c is a side perspective view of an alternative embodiment with spars situated on an internal portion of the inner body.

### DETAILED DESCRIPTION

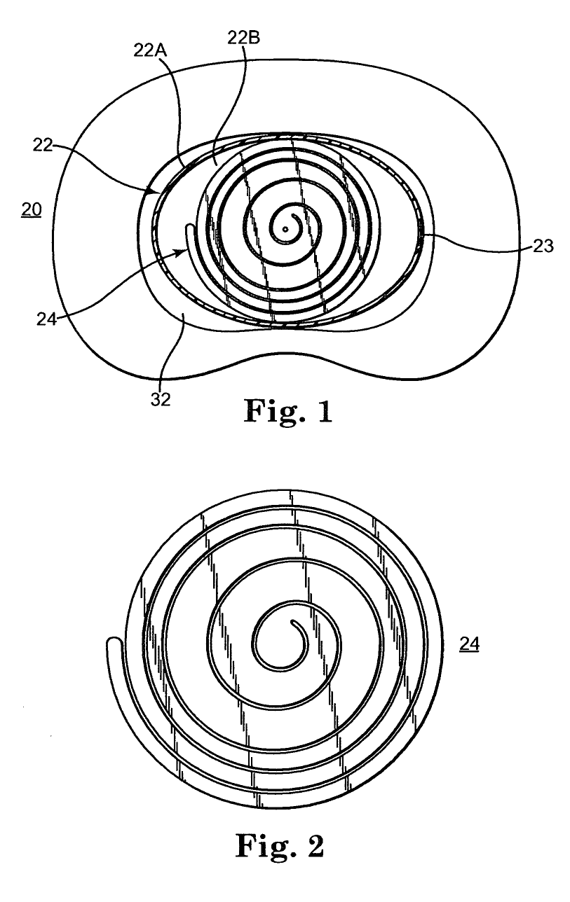

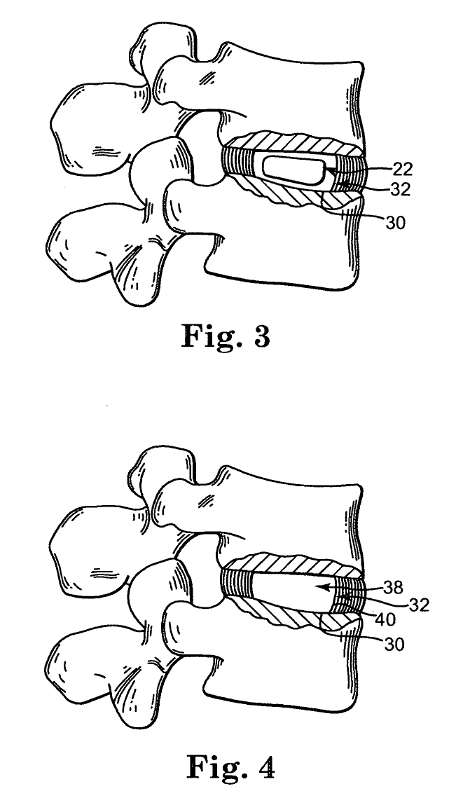

With reference to FIGS. 1-3, the present invention is a spinal stabilization system 20 for insertion into an intervertebral disc of a vertebra. The spinal stabilization system 20 is intended for the improvement, augmentation, or replacement of a damaged intervertebral disc nucleus 32 (nucleus pulposus). The spinal stabilization system 20 may stabilize the intervertebral disc nucleus 32 while at the same time relieving pain. The spinal stabilization system 20 may improve upon 1) implant expulsion or extrusion; 2) implant sizing; 3) implant conformity; and 4) reduction or prevention of bone edema that may be part of an inflammatory response. The spinal stabilization system 20 may also be referred to by such names as a prosthetic nucleus, a prosthetic implant, an intervertebral disc implant, and a nucleus pulposus implant.

One embodiment of the present embodiment spinal stabilization system 20 includes a polyvinyl alcohol (PVA) hydrogel bag 22 and a spiral implant 24 wherein the spiral implant 24 is positioned into the PVA hydrogel bag 22 after the PVA hydrogel bag 22 is inserted into the spine. The PVA hydrogel bag 22 includes an outer wall 22A (or skin) of PVA hydrogel material forming a substantially hollow interior 22B. The PVA hydrogel bag 22 further includes an opening 23. The opening 23 may be any desired size and shape that allows it to receive the spiral implant 24. The term “bag” is utilized herein to describe the structure formed from the PVA hydrogel because it includes an opening 23 and a hollow interior 22B formed by a wall 22A. Other terms may be utilized to describe the structure formed by the PVA hydrogel, such as, but not limited to, bladder, pouch, purse, sack, etc.

The PVA hydrogel bag 22 may be made in a variety of sizes and shapes in order to optimally fit the intervertebral disc nucleus 32 and to accept spiral implants 24 of different sizes. The “bag” shape may be any shape when dehydrated or when hydrated, such as, but not limited to round, disc, circular disc, egg, oblong, elliptical, etc., and include surfaces that are flat, concave, or convex, depending on the needs of those skilled in the art and the anatomy of each patient. Additionally, the wall 22A of the PVA hydrogel bag 22 can be made of a desired thickness and may be uniform or variable thickness. Variations in thickness of the wall 22A of the PVA hydrogel bag 22 may contribute to the load bearing capacity of the PVA hydrogel bag 22 and the entire spinal stabilization system 20. When the hydrogel material becomes hydrated the wall 22A typically becomes thicker. Such changes in the wall 22A and PVA hydrogel bag 22 can be accounted for before insertion of the spinal stabilization system 20. The PVA hydrogel bag 22 is preferably of a size and shape to contain the spiral implant 24 without leaving extra room for the spiral implant 24 to float or migrate around the interior of the bag.

The PVA hydrogel bag 22 may be formed by a casting or a dip coating procedure. Such procedures may utilize a collapsible bladder or a casting mold for forming hydrogel into the desired shape with the desired wall 22A thickness. The hydrogel may then be cross-linked using processes known to those of skill in the art. In one embodiment the hydrogel may be cross-linked using known chemical cross-linking agents. In one alternative embodiment, the hydrogel may be physically cross-linked through a freeze and thaw process. The method of forming the PVA hydrogel bag 22 does not affect the present invention.

The nature of the materials employed to form the PVA hydrogel bag 22 are preferably selected so that the formed implants have sufficient load bearing capacity for the application. A compressive modulus of at least about 0.1 Mpa is desired, although compressive modulus in the range of about 0.1 Mpa to about 20 Mpa may be preferred. In addition, a variety of methods have been reported to enhance the mechanical strength of PVA hydrogel. The formation of hydrogel materials that may be useful in the present invention have been previously reported in U.S. Pat. Nos. 4,663,358, 5,047,055, 5,534,028, 6,280,475, and 5,705,780, which are each incorporated in their entirety for all that they teach and disclose.

The spiral implant 24 is an intervertebral implant preferably including an elongated elastic body that takes on the form of a spiral in the force free state. The spiral implant 24 consists preferably of a plastic such as polyurethane. The deformation properties of the material are preferably similar to those of the intervertebral disc and may include a Shore hardness range from about 70 A to 90 A, preferably around 80 A. The material of which the elastic body is manufactured may correspond to the elastic properties of an intervertebral disc, such as, for example polyurethane or polyurethane-silicon mixtures. In further embodiments different characteristics of different materials may be utilized, such as, softer or more springy materials. The spiral implant 24 can fill differing diameters by cutting the spiral to the desired length such that the length will form the desired number of turns and therefore the desired diameter. In addition, the spiral implant 24 can be unwound and therefore inserted in a minimally invasive manner such that it forms a larger shape after placement.

The height of the spiral implant 24 may be consistent along the length of the spiral, may increase continually in either direction, or may have any of a number of height and width profiles. The overall height of the spiral implant 24 length may be between about 4 mm and about 10 mm. In alternative embodiments, the thickness of the elastic body that forms the spiral may vary between the interior loops and the exterior loops. The coiled height may also be adapted to the individual curvature of the vertebral surfaces. The spiral implant 24 may have a diameter in the range of 20 to 30 mm or preferably in the range from about 23 to about 25.5 mm. The spiral implant 24 may also be clipped to customize the length. The spiral implant 24 can be adjusted intra-operatively to the desired size through the number of turns formed in the PVA hydrogel bag 22. Such procedures for adjusting the length of the spiral implant 24 are known in the art. Adjusting the height or the length of the spiral implant 24 allows the present invention to be customized to different patients.

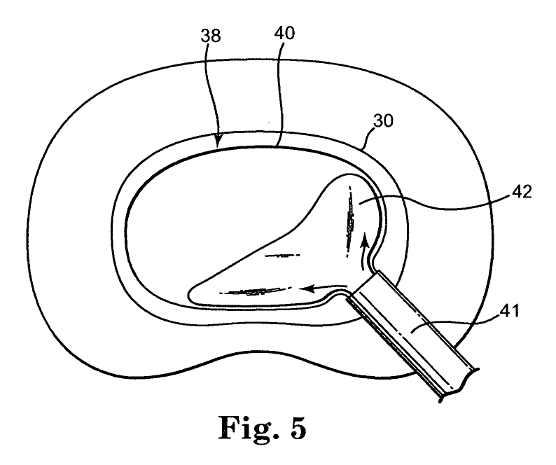

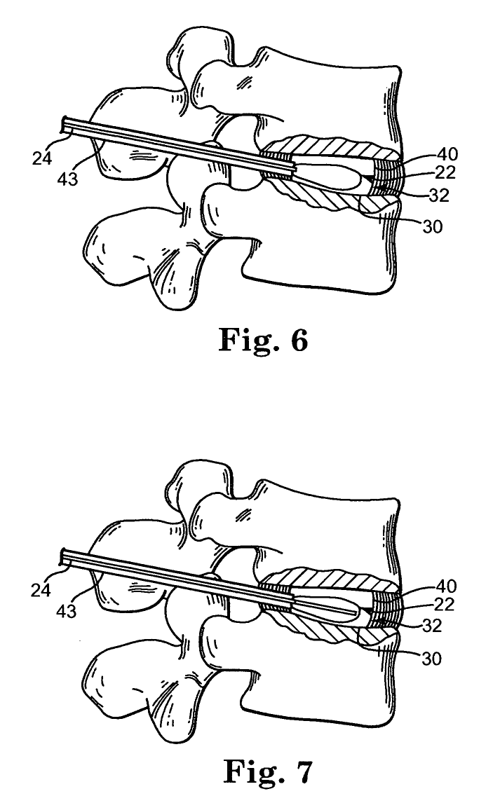

With additional references to FIGS. 4-7, the insertion of the present invention spinal stabilization system 20 will now be herein described. The present description for inserting the spinal stabilization system 20 is described as a posterolateral procedure. In other embodiments the stabilization system may be placed using a lateral percutaneous approach to the disc space 30. In still further embodiments, the present invention may be implanted from other approaches, such as lateral or anterior or posterior such as used for microdiscectomy. The PVA hydrogel bag 22 may be placed into the cavity 38 in a dehydrated or hydrated state depending on the preference of the person performing the insertion.

A disc space 30 will be typically first prepared by removing a desired amount of the intervertebral disc nucleus 32 from the intervertebral space during a nuclectomy. The nuclectomy may be performed using known surgical instruments and methods through a small annulotomy. The removal of the disc nucleus 32 creates a cavity 38 of a desired shape and size. The cavity 38 may be in a desired position and orientation. In some embodiments only a portion of the disc nucleus 32 may be removed. In further embodiments the entire disc nucleus 32 may be removed depending on the specific needs of the patient. In the present embodiment, as much of the disc nucleus 32 may be removed as possible without damaging the cartilaginous endplates. As the disc nucleus 32 is removed, the size and shape of the cavity 38 being formed can be repeatedly checked utilizing a contrast solution or other means known in the art. The cavity 38 may be made in a minimally invasive manner such that a relatively small incision is made in the patient. The annulus may be left substantially intact.

When fully formed by removing the desired portion of the disc nucleus 32, the cavity 38 may have a generally circular disc shape. In further embodiments, the eccentricity ratio (major axis over minor axis) of the cavity 38 may vary from about 1.0 to about 1.6. A wall 40 of the cavity 38 may have a smooth surface and a regular geometry, but can be any shape, size, or form desired. In addition, disc fragments in the intervertebral space may also be removed.

The size and geometry of the cavity 38 may also be evaluated intraoperatively using a balloon 42 inserted on a catheter 41, or, in alternative embodiments, through an annula. The balloon 42 may be mounted on a catheter 41 or other inflation device and placed into the cavity 38. Filling the balloon 42 with a contrast solution allows the cavity 38 to be visualized using a fluoroscope. The volume and dimension of the cavity 38 may be approximated by measuring the volume of the contrast solution utilized to fill the balloon 42 or by looking at the fluoroscope. This information may be used to select the PVA hydrogel bag 22 and the spiral implant 24 such that the combination of the two reasonably approximates the volume of the cavity 38. The balloon 42 may then be emptied of contrast solution and removed.

In still further embodiments, a cannula 43 may be utilized as an aid to insertion. The PVA hydrogel 22 bag may be compressed, folded, or otherwise reduced in size to pass through the cannula 43. The PVA hydrogel bag 22 may also be passed through the delivery cannula 43 utilizing a blunt stylet to push the PVA hydrogel bag 22 into the cavity 38. The blunt stylet may be inserted into the hollow interior 22B through the opening 23 in the PVA hydrogel bag 22 during insertion such that the opening 23 remains properly oriented relative to the delivery cannula 43 for later insertion of the spiral implant 24. The stylet may then be utilized to place the PVA hydrogel bag 22 into the desired position. The position may be confirmed using known imaging techniques. In other embodiments, an insertion device that includes a force transmitting element may be used such as reported in U.S. Pat. No. 5,800,549, which is incorporated herein by reference for all that it teaches and discloses.

The dimensions of the PVA hydrogel bag 22 may vary depending on each particular case. The PVA hydrogel bag 22 is preferably wide enough to entirely support the adjacent vertebrae and is of a height sufficient to separate and support the adjacent vertebrae after the insertion of the spiral implant 24. The volume of the PVA hydrogel bag 22 may be as large as about 99% of the volume of the intervertebral disc space 30 when combined with the spiral implant 24.

The PVA hydrogel bag 22 may then be hydrated by adding fluid. Alternatively, the PVA hydrogel bag 22 may be exposed to body fluids in situ, causing the PVA hydrogel to absorb water and swell. As may be appreciated, the PVA hydrogel bag 22 may be exposed to hydration fluids at any time during the process. The spiral implant 24 may then be deployed into the PVA hydrogel bag 22.

The spiral implant 24 may be deployed into the cavity 38 of the intervertebral disc space 30 and into the PVA hydrogel bag's 22 hollow interior 22B utilizing devices known in the art. Such techniques are more completely described in the patents that disclose the features of the spiral implant 24, which were previously incorporated by reference. A spiral implant 24 with the desired physical characteristics may be first selected. The selected spiral implant 24 is then deployed until it fills a desired amount of the PVA hydrogel bag 22. In the present embodiment the spiral implant 24 may be deployed through the cannula. The end of the spiral implant 24 may then be trimmed if it has not been done previously. The combined volume of the expanded (hydrated) PVA hydrogel bag 22 and the spiral implant 24 should approximate the nucleus cavity 38 volume.

In one embodiment, the spiral implant 24 can be drawn into the insertion instrument by reverse winding. The insertion instrument may be only insubstantially larger in the insertion region than the cross-section of the elongated elastic body. Examples of delivery instruments useful for inserting the spiral implant 24 are taught and disclosed by U.S. Pat. Nos. 6,165,218, 5,919,235, 5,800,549 and 5,716,416 which are incorporated by reference herein for all that they teach and disclose. The PVA hydrogel bag 22 may help to protect the endplate from damage during the insertion of the spiral implant 24.

Next, the excess material from the spiral implant 24 may be removed by cutting. The spiral implant 24 may also be checked to confirm that it remains in position.

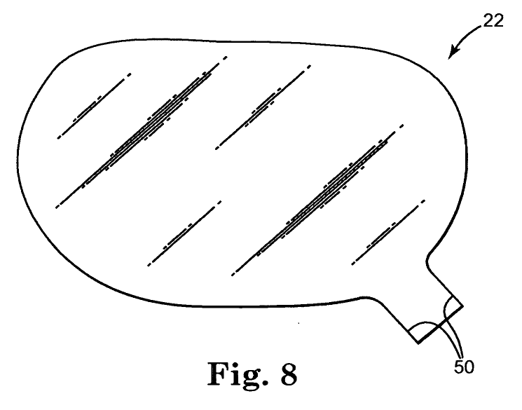

As illustrated in FIG. 8, a closure 46 may be then utilized to close the PVA hydrogel bag 22 and secure the spiral implant 24 in the hollow interior 22B of the PVA hydrogel bag 22. In the illustrated embodiments, the closure 46 may be two or more tethers 50 that are integrally formed as part of the wall 22A of the PVA hydrogel bag 22. The tethers 50 may be simply tied together to seal the PVA hydrogel bag 22 opening 23. Alternatively or in addition, closure ties or strings may be integrated as closure 46 into the wall 22A of the PVA hydrogel bag 22 when the bag is formed. Other closure means may include inter-locking latches, adhesives, or other fibers integrated into the PVA hydrogel bag 22 and that can be tied, such as including a draw string. Closure ties may be made of a Dacron™ or another suitable material and may be integral to the perimeter of the opening 23 in the PVA hydrogel bag 22. Such ties or strings may be tied, drawn closed, or pulled tight like a purse string. In addition, closing the opening 23 of the PVA hydrogel bag 22 may be accomplished by stitching. In still further embodiments the closure may be a mechanical type closure integrated into the PVA hydrogel bag 22 structure. Such closures may include valves, like check valves, duckbill valves, plugs, screw caps, flapper valves, etc. Such devices may be incorporated into the PVA hydrogel bag 22 by sonic welding, stitching, or other means known to those in the art.

Excess portions of the closure device 46, whether part of the wall 22A of the PVA hydrogel bag 22 or integrated ties or strings, may be removed by clipping. The bag closure 46 may contribute to the large implant cross-sectional area of the spinal stabilization system 20. Finally, the surgical site is closed in a typical manner.

The PVA hydrogel bag 22 will expand when completely hydrated to substantially or completely fill the portion of the nucleus cavity 38 not occupied by the spiral implant 24. Moreover, the spiral implant 24 may force the PVA hydrogel bag 22 outward, pushing the wall 22A into the surface of the disc cavity 38. In order to check on the position of the spiral implant 24 during and after insertion, X-ray-positive markers may be situated on, along, or within the elastic body. Barium, tantalum or the like can be used as a marker material. Once the PVA hydrogel bag 22 is tied shut, the PVA hydrogel bag 22 and the spiral implant 24 may act as one integral unit.

In one alternative embodiment, a dilation balloon may be placed inside of the PVA hydrogel bag 22 before placement of the PVA hydrogel bag 22 into the cavity 38. The dilation balloon may be of a desired shape and size such that once the dilation balloon and the PVA hydrogel bag 22 are placed into the cavity 38, the dilation balloon may be inflated to deploy the PVA hydrogel bag 22 into position. The PVA hydrogel bag 22 may be fit over the dilation balloon and then the two may be folded into a desired size and shape for placement through the delivery cannula 43. In further embodiments, the PVA hydrogel bag 22 may simply be placed over the dilation balloon and the combination of the two placed through the delivery catheter 41 without folding. Again, a stylet may be utilized to push the PVA hydrogel bag 22 and the dilation balloon through the delivery cannula 43.

Once the PVA hydrogel bag 22 and the dilation balloon are inserted into the cavity 38, the PVA hydrogel bag 22 and the dilation balloon may be observed with known techniques to help properly position the PVA hydrogel bag 22. As may be appreciated, radio opaque medium may be used to expand the dilation balloon so it can be more easily observed. Adjustments may be done as necessary and the dilation balloon can be expanded to place the PVA hydrogel bag 22 into position. After the PVA hydrogel bag 22 is inserted into the cavity 38 and placed into position by the expansion of the dilation balloon, the dilation balloon may be removed. The dilation balloon may be removed by first removing the expansion medium to deflate the balloon.

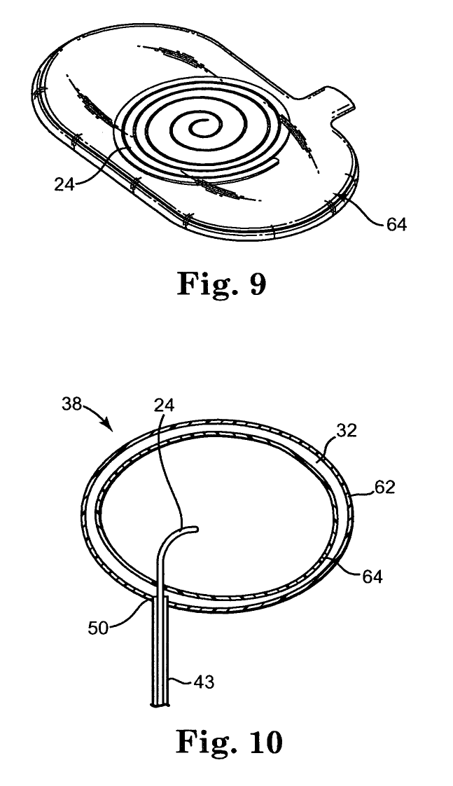

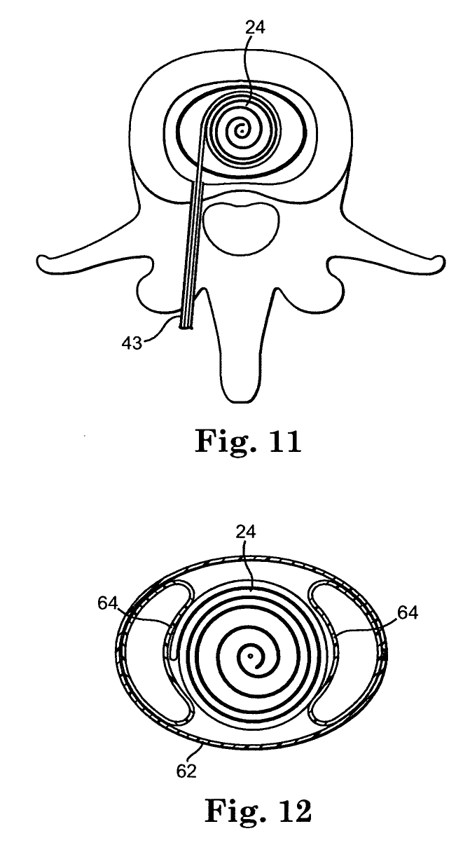

In one alternative embodiment illustrated in FIGS. 9-11, the stabilization system 20 may include two or more separate structures, such as an outer bag 62 and an inner body 64. The outer bag 62 may be formed of the same or different material than the inner body 64. Preferably the outer bag 62 is made of a material resistant to wear such as a textile material. The textile material of the outer bag 62 may be woven, knitted, or braided. In addition, the material may be Kevlar, ultra high molecular weight polyethylene, polyester (such as Dacron™), or any other biocompatible material useful for forming structures for placement in the body and resistant to wear.

The inner body 64 is contained inside of the outer bag 62. However, as further explained below, the inner body 64 may be outside the circumference of the spiral implant 24. The inner body 64 may be made of the same or similar material, but may also be a substantially different material, such as a hydrogel or a resilient hydrogel or elastomer. In addition, the inner body 64 may be made of a more solid material, like a foam or a closed cell foam, such as polyurethane. In further embodiment, the inner body 64 may be substantially one piece or may be two or more pieces.

The outer bag 62 may completely surround the inner body 64. In the present embodiment the inner body 64 is placed into the outer bag 62 and is held in place by the complimentary shape the inner body 64 has with the outer bag 62. In further embodiments the outer bag 62 and the inner body 64 may be physically fitted, integrated, or attached in any manner known to those in the art, such as, for example, sewing, chemical bonding, or by forming the outer bag 62 and the inner body 64 as one integral structure. As further discussed below, in still further embodiments the inner body 64 may be fitted with other structures. The more resilient material of the outer bag 62 as compared to the PVA hydrogel of the embodiment previously described may allow the stabilization system 20 to have a longer useful life. The inner body 64 may be compressed or folded for delivery through the bore of a cannula 43 or through a narrow hole in the annulus, whether separately from the outer bag 62 or already placed in the outer bag 62. As may be appreciated, the size of the outer bag 62 and the inner body 64 may be selected in advance for the cavity 38.

Utilizing a stabilization system 20 with outer bag 62 and inner body 64 may allow the stabilization system 20 to better spread the force transferred on the vertebral bodies evenly throughout the disc space 30. Because the spiral implant 24 normally supports most of the load placed on the stabilization system 20, that area of the spine covered by the spiral implant 24 receives most of the force normally spread throughout the entire disc nucleus 32. When the inner body 64 is placed in the outer bag 62 along with the spiral implant 24, the inner body 64 transfers some of the load outside the circumference of the spiral implant 24. In addition, transferring the load outside of the circumference of the spiral implant 24 reduces the wear on the outer bag 62. In further embodiments more than one inner body 64 may be placed into the outer bag 62 along with the spiral implant 24 to form a shape that compliments and fills the outer bag 62. Filling the outer bag 62 with the inner body 64 and spiral implant 24 such that they substantially fill the outer bag 62 and form a complimentary shape to the outer bag 62 may further increase the resistance to expulsion of the spinal stabilization system 20.

In other aspects of the invention, kits designed for forming the spinal stabilization system 20 may be provided. In one form, a kit may include a load bearing spiral implant 24 along with the PVA hydrogel bag 22. Other kits may include a variety of different PVA hydrogel bags 22 and spiral implants 24 for custom fitting. Such kits may further or alternatively include an outer bag 62, an inner body 64, and a spiral implant 24 such that during implantation a surgeon would place the outer bag 62 and fill the outer bag with the inner body 64 and the spiral implant 24 to form a shape that compliments and substantially fills the interior cavity of the bag 62. In various embodiments the spiral implant 24 may be placed inside the outer bag 62 inside a space formed by the inner body 64 (FIG. 9) or between two inner bodies 64 that compliment the shape of the outer bag 62 and the spiral implant 24 (FIG. 12).

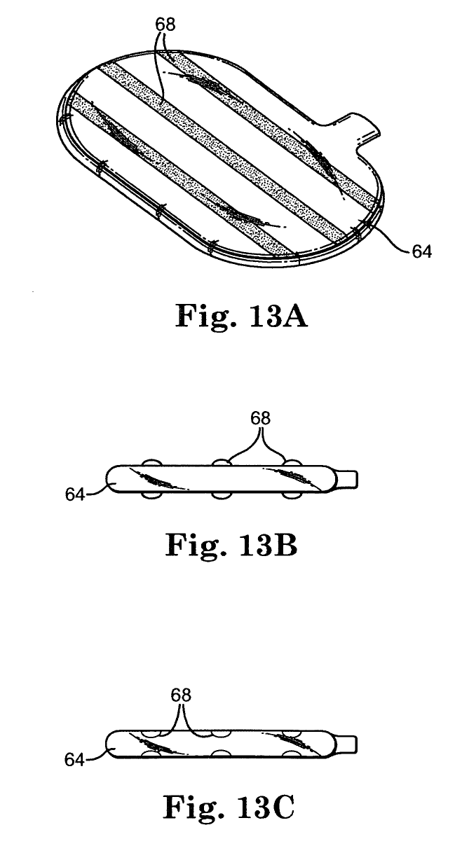

The stabilization system 20 may further include additional features that provide additional force distribution and structural stability. One additional feature may include a supporting structure 66. As illustrated in FIGS. 13a-c, in one embodiment the support structure 66 may be spars 68 that extend laterally to connect different portions of the inner body 64. The spars 68 may extend either over or over and under the area of the outer bag 62 where the spiral implant 24 is to be placed. The spars 68 may help prevent the spiral implant 24 from overlapping or migrating over or under the inner body 64. The spars 68 may be integrated with the inner body 64 or may be completely separate structure from the inner body 64. In FIG. 13b the spars 68 are situated on the outside of the inner body 64. In FIG. 13c the spars 68 are situated on the inside of the inner body 64. In a further embodiment, where the inner body 68 includes two separate structures, the spars 68 may run substantially between the inner bodies 64 so as to provide structural stability while at the same time providing the proper placement and orientation of the inner bodies 64. These spars may aid in dissipating or distributing the force exerted by the stabilization system 20 on the adjacent vertebral bodies more evenly or over a larger intervertebral space. In alternative embodiments (not shown) the spars 68 may be furthermore integrated with the outer bag 62, placed inside the outer bag 62, or integrated into or outside of the outer bag 62. In still further embodiment there may be only one, or more than one spar 68 incorporated. Moreover, the spars may take on a variety of sizes and shapes.

They stabilization system 20 including the outer bag 62 and the inner body 64, and in some embodiments, spars 68, may be placed into the cavity 38 created in the intervertebral space substantially in the same manner as previously described. The supporting structure, spars 68, and the outer bag 62 and inner body 64 may be compressed, folded, or otherwise reduced in size to pass through the cannula 43 and into the intervertebral space.

The combination of the PVA hydrogel bag 22 or outer bag 62 and inner body 64 and spiral implant 24 may create a spinal stabilization device 20 that has a greater resistance to extrusion than the components would if implanted alone. The combination with the spiral implant 24 provides for a body that acts in coordination to create a body with a larger footprint so as to reduce the likelihood that the stabilization system 20 will be expelled from the intervertebral disc space 30. Put another way, the spiral implant 24 anchors the PVA hydrogel bag 22 in place and the combination of the two helps to prevent expulsion as compared to a solid PVA hydrogel insert. The solid PVA hydrogel insert's compliance under pressure allows for expulsion but the stiffening effect of the spiral implant 24 helps to prevent such expulsion.

Proper spinal stabilization system 20 sizing and conformity helps to prevent over pressurizing the vertebral endplates. The stabilization system 20 that includes the PVA hydrogel bag 22 further helps to provide the proper fit and conformity because of its ability to swell (hydrate). Moreover the PVA hydrogel bag 22 or outer bag 62 may provide a low modulus transition layer between the spiral implant 24 and the endplates. In other words, the PVA hydrogel bag 22 or 62 may provide a deformable transition layer between the spinal endplates and the relatively deformation resistant spiral implant 24. The PVA hydrogel bag's 22 or 62 ability to custom fit the nucleus cavity 38, and provide a transition layer between the spiral implant 24 and the endplates, may reduce or prevent inflammatory processes that lead to bone edema and potentially to bone resorption and remodeling. The PVA hydrogel bag 22 or 62 also helps for distribution of the force between the spiral implant 24 and the end plates of the spine.

The present invention spinal stabilization system 20 may also help to reduce bone edema. Bone edema is a potential difficulty with nucleoplasty because the vertebral endplates may be exposed to lower than normal intradiscal pressures during the degenerative disc disease process. The endplates may adapt to the lower pressure and loose some strength and rigidity, and, thus, their load bearing capability as expressed by Wolf's law. Bone edema may result from pressure necrosis associated with a nucleus implant that restores loading on the endplates adapted to the lower pressure degenerative condition.

Some nucleus implants may also create too much pressure on the vertebral endplates and deform the endplates to the point where an inflammatory process begins. The inflammatory process may set up in the bone and result in the edema. A mild inflammatory reaction may occur if the resting contact pressure inside the disc cavity is greater than the resting intradiscal pressure of a healthy disc. A severe inflammatory reaction may then occur if the pressure is great enough to deform the vertebral endplates. Over deformation of the endplates may produce erosion of the endplate cartilage and microfracture of the endplates and the underlying cancellous bone.

Severe pressure necrosis and the ensuing inflammatory response may also lead to bone resorption and remodeling. The resorption and remodeling process may result in fibrous tissue being deposited between the implant and the bone or the cancellous bone can remodel with substantial fat or marrow deposits replacing the cancellous bone. The PVA hydrogel bag 22 or 62 of the spinal stabilization system 20, however, may act as a transition zone between the implant and the bone to allow a favorable load or pressure distribution within the implanted disc.

In one alternative embodiment, the disc space 30 may be distracted to a desired level before insertion of the spinal stabilization system 20. The appropriate size of the PVA hydrogel bag 22 or 62 and the spiral implant 24 desired in a particular case may be determined by distracting the disc space 30 to a desired level after the nuclectomy and measuring the volume of the distracted space.

In other aspects of the invention, kits designed for forming the spinal stabilization system 20 may be provided. In one form, a kit may include a load bearing spiral implant 24 along with the PVA hydrogel bag 22 or 62. Other kits may include a variety of different PVA hydrogel bags 22 or 62 and Spiral implants 24 for custom fitting.

In still further embodiments, the spinal stabilization system 20 may deliver desired pharmacological agents, such as a growth factor. The pharmacological agent may also be one used for treating various spinal conditions, including degenerative disc disease, spinal arthritis, spinal infection, spinal tumor and osteoporosis. Such agents include antibiotics, analgesics, anti-inflammatory drugs, including steroids, and combinations thereof.

The pharmacological agents are preferably dispersed within the hydrogel for in vivo release. The pharmacological agents may be dispersed in the implants by adding the agents to the solution used to form the implant, by soaking the formed implant in an appropriate solution containing the agent, or by other appropriate methods known to the skilled artisan.

Various modifications and additions may be made to the exemplary structures and steps discussed. Various combinations, permutations, and rearrangements of those structures and steps may similarly be made without departing from the scope of the present invention. Accordingly, the scope of the present invention is intended to embrace all such alternatives, modifications, and variations as fall within the scope of the claims, together with all equivalents thereof.

1. A spinal stabilization system for insertion into a cavity formed by the removal of a desired amount of a nucleus in an intervertebral disc comprising:
a bag; and
a spiral implant implanted into the bag.

2. The spinal stabilization system of claim 1 wherein the combination of the spiral implant and the bag is approximately the size of the cavity.

3. The spinal stabilization system of claim 1 further comprising a dilation balloon for insertion into the bag.

4. The spinal stabilization system of claim 1 wherein the bag is formed of a hydrogel.

5. The spinal stabilization system of claim 1 wherein the spiral implant is a shape memory plastic.

6. The spinal stabilization system of claim 1 wherein the bag includes an opening for receiving the spiral implant into the bag.

7. The spinal stabilization system of claim 6 wherein the bag further comprises a closure selected from the group consisting of ties, strings, tethers, or stitches.

8. The spinal stabilization system of claim 6 wherein the bag further comprises a mechanical closure.

9. A method for repairing a damaged intervertebral disc comprising:
removing a desired portion of a nucleus of the intervertebral disc to form a cavity;
inserting a PVA hydrogel bag into the cavity, the PVA hydrogel bag including a wall forming an interior and an opening;
inserting a spiral implant into the interior of the PVA hydrogel bag through the opening; and
closing the opening of the PVA hydrogel bag.

10. The method of claim 9 further comprising hydrating the PVA hydrogel bag after insertion into the cavity.

11. The method of claim 9 wherein inserting the PVA hydrogel bag includes inserting the PVA hydrogel bag in a hydrated state.

12. The method of claim 9 wherein inserting the PVA hydrogel bag and inserting the spiral implant further comprise inserting the PVA hydrogel bag and the spiral implant through a cannula.

13. The method of claim 9 wherein removing a portion of the cavity further comprises removing a portion of the cavity in a minimally invasive manner

14. The method of claim 9 further comprising:
measuring the cavity to determine the size and shape of the cavity; and
selecting the PVA hydrogel bag to fit the size and shape of the cavity.

15. The method of claim 14 wherein measuring the cavity further comprises
inserting a balloon into the cavity; and
filling with a contrast solution and visualizing with a c-arm.

16. The method of claim 9 further comprising:
inserting a dilation balloon into the PVA hydrogel bag before inserting the PVA hydrogel bag into the cavity; and
inflating the dilation balloon to deploy the PVA hydrogel bag after the PVA hydrogel bag and dilation balloon have been inserted into the cavity.

17. An intervertebral disc nucleus pulposus implant, comprising:
a PVA hydrogel bag sized for introduction into a cavity created in an intervertebral disc space, the PVA hydrogel bag including a closeable opening in the bag; and
a spiral implant for insertion into the PVA hydrogel bag after the PVA hydrogel bag is inserted into the cavity in the intervertebral disc space.

18. A spinal stabilization system comprising:
a bag;
an inner body for placement inside of the bag; and
a spiral implant for insertion into the bag.

19. The spinal stabilization system of claim 18, wherein said inner body comprises a low modulus elastomer, a foam material, a hydrogel, a ribbed structure, or a combination thereof.

20. The spinal stabilization system of claim 18 wherein the bag is a textile material.

21. The spinal stabilization system of claim 20 wherein the textile material of the bag is selected from the group consisting of woven, knitted, or braided.

22. The spinal stabilization system of claim 18 wherein the spiral implant is inserted into the inner body.

23. The spinal stabilization system of claim 18 wherein the inner body and the spinal implant form a complimentary shape that substantially fills the inside of the bag.

24. The spinal stabilization system of claim 18 further comprising at least one spar extending laterally inside the bag wherein the spar provides further structural support to the system and helps to retain the spiral implant in position relative to the inner body.

25. The spinal stabilization system of claim 18 further comprising at least one spar integrated with the bag wherein the spar provides further structural support to the system and helps to retain the spiral implant in position.

26. The spinal stabilization system of claim 18 wherein the bag is made of a flexible polymer.

27. A method for replacing nucleus of an intervertebral disc, said method comprising:
removing a desired portion of an intervertebral disc nucleus from a patient thereby creating a cavity within the annulus fibrosus;
inserting a prosthetic bag into the cavity using a cannula, the bag including an inner cavity and an opening;
placing one or more bodies into the bag;
positioning a spiral implant into the bag; and
closing the opening using a closing mechanism.

28. The method of claim 27, wherein said step of inserting the prosthetic bag to the annulus cavity further comprises:
placing a dilation balloon into the cavity of the bag; expanding the dilation balloon.

29. The method of claim 27 further comprising inserting spars into the bag wherein the spar helps to retain the spiral implant in position relative to the one or more bodies.

30. The method of claim 27 further wherein inserting the prosthetic bag further comprises inserting a prosthetic bag that releases a pharmacological agent.
